(19) United States
(12) Patent Application Publication
(10) Pub. No.: US 2022/0084239 A1
(43) Pub. Date: Mar. 17, 2022

(54) EVALUATION OF AN ULTRASOUND-BASED INVESTIGATION

(71) Applicants: Gerd BODNER, Vienna (AT); Karl CERNY, Vienna (AT); Manfred GREIFONER, Vienna (AT); Christopher PIVEC, Vienna (AT)

(72) Inventors: Gerd BODNER, Vienna (AT); Karl CERNY, Vienna (AT); Manfred GREIFONER, Vienna (AT); Christopher PIVEC, Vienna (AT)

(21) Appl. No.: 17/024,490

(22) Filed: Sep. 17, 2020

(51) Int. Cl. G06T 7/73; G16H 50/20; G16H 30/40; A61B 8/00

(52) U.S. Cl. CPC G06T 7/73; G16H 50/20; G16H 30/40; A61B 8/4263; H04N 5/2253; G06T 2207/30204; G06T 2207/10132; G06T 2207/30196; G06T 2207/10016

(57) ABSTRACT

Proposed are a device and a method for supporting an evaluation, documentation and categorization of an ultrasound-based examination. The method comprises the steps:
carrying out the ultrasound-based examination by means of a transducer at a point in time,
determining information representing a location and/or position of the transducer at the point in time and
automatic allocating of an image, recorded at the point in time by means of the transducer to the information representing the location and/or position of the transducer at the point in time.

### TECHNICAL FIELD

The present invention relates to a device and a method for assisting the evaluation, automated documentation and categorisation of collected data of an ultrasound-based examination, in particular of an ultrasound-based examination result.

### BACKGROUND AND SUMMARY

Here, the ultrasound-based examination in particular as the ultrasound-based generation of an image (or video) of an ultrasound examination point (or several points) of a part of a patient's body or respectively the result of the examination, is to present a documentation of the ultrasound examination point (or several points) on the patient's body by ultrasound-based images. The evaluation in the sense of the present invention may be the exact reproduction of the transducer position for the interpretation of the ultrasound images.

Ultrasound-based examinations are known in the prior art. Their advantage compared to electromagnetic or X-ray-based methods consists in particular in a non-present magnetic field exposure and/or radiation exposure of the patient compared to X-ray-based methods such as CT and X-rays, and equipment which is distinctly more economical and space-saving. It is estimated that 50% of current musculoskeletal magnetic resonance tomography, MRT, examinations can basically also be covered by high resolution ultrasound. This is also due to the fact that the detail density (spatial resolution) of high resolution ultrasound is far higher than that of MRT and CT. In particular, the recording of small nerves, tendons, ligaments and other soft tissues can be achieved better or at least equivalent to high quality MRT.

Owing to the visual representation of the ultrasound image of the ultrasound examinations which is less able to be interpreted intuitively compared to MRT/CT, many ultrasound examination results are esteemed less, or not at all, by the specialists. As a result, many specialists have difficulties in the interpretation of the ultrasound images. This goes so far that the specialists generally can not even recognize in which position the imaged part of the body has been situated with respect to the ultrasound transducer (also “Transducer”) during the photography or respectively which part of the body is concerned at all. In order to improve the usability of the ultrasound images, usually therefore a time-consuming documentation of the individual views is carried out, in order to be able to retrospectively allocate the created ultrasound image to the examined point on the body. When this documentation also does not assist the specialist further, the ultrasound-based examination is sometimes requested again by the specialist or is carried out by him/herself.

This leads to an increased expenditure in terms of time and hardware, delayed diagnoses with the corresponding consequences, and increased costs for the health system.

WO 98/08112 shows a method for the electromagnetically supported creation of 3D models of the human body, in which an allocation of ultrasound images with respect to one another takes place, by the transducer emitting electromagnetic waves for determining position, so that individual ultrasound cross-sectional images can be used for creating the 3D model.

Proceeding from the above-mentioned prior art, it is an object of the present invention to make the interpretability of results of ultrasound-based examinations more precise and easier, and thereby to lead to a generally greater acceptance. This leads to a significant cost reduction for the patient and the health system and to a prevention of delayed diagnosis with all its medical and economic consequences. For the user carrying out ultrasound, the inventive presentation leads inter alia to a minimizing of documentation errors and to a distinct saving of time.

### DISCLOSURE OF THE INVENTION

The above-mentioned problem is, according to the invention, solved by a method for assisting a recording, evaluation and automatic categorisation of an ultrasound-based examination.

The ultrasound-based examination can take place on the body of a patient.

In particular, the support in the interpretation (evaluation) of the images created on the basis of ultrasound takes place by third parties.

In a first step, the ultrasound-based examination is carried out by means of a transducer at a first point in time.

In other words, ultrasound recordings are generated by means of the transducer, wherein the transducer is placed onto the surface of the patient's body. Subsequently, information representing a site and/or position of the transducer at the point in time is determined.

In other words, it is documented in what manner the transducer was situated or respectively oriented while the ultrasound-based examination took place.

In particular, the determining of the information (non-moving and moving images (e.g. a photograph=photography) or other image material) can take place automatically.

In other words, it is automatically documented in which relative location and/or position of the transducer with respect to the body (part) of the patient the ultrasound image material was recorded at the point in time.

Subsequently, an automatic allocation takes place of an image, recorded at the point in time by means of the transducer, to the information, which represents the position/location of the transducer at the point in time.

Here, for example, a time stamp of the image material recorded on the basis of ultrasound, and a time stamp of the information which designates the position/location of the transducer, can be automatically compared with one another. For example, a file or a dataset can be created, in which the information and the image material, determined on the basis of ultrasound, are stored in such a way that on retrieval of the dataset, a simultaneous presentation of the information and of the ultrasound-based examination material can take place.

For example, a combined image file can comprise the information and the ultrasound image material. The image material can comprise a still shot (“photo”) and/or a moving image sequence (“video”).

Subsequently, a presentation can take place directly of the data material assigned to one another.

For example, the presentation can take place for a user and/or third party (e.g. the person requesting the examination) of the ultrasound apparatus and/or for the patient, in order to view the results of the ultrasound-based examination and to ensure a correct documentation.

For example, it can be checked here whether the information representing the location and/or position of the transducer is able to be interpreted sufficiently easily or respectively is suitable.

Alternatively or additionally, the information items which are assigned to one another can be transmitted by means of a remote transmission to remote positions (in the sense of teleradiology).

This can take place in particular in real time. In this way, specialists/experts/third parties present at the remote position can be involved in the interpretation of the ultrasound-based examination results and—if desired—can deliver their assessment/diagnosis in real time.

In particular, for this they can recommend or request corrections to the location/position of the transducer in real time.

These can be directed to the user, the patient or a motor (a software, which controls a “robot”), via which e.g. remotely the relative position between the transducer, the sensor used for determining the information with respect to its location and/or position, and/or the patient (e.g. by means of possibility of sitting and/or lying, adjustable by motor) can be taken.

By the information items which are assigned to one another, an easier and more reliable interpretation of the results of the ultrasound examination can take place.

Alternatively or additionally, the data material assigned to one another can be stored on a local data memory and/or on a network memory (in a cloud), in order to document the ultrasound examination for a later inspection.

As a result, the risk of requiring a second ultrasound examination is reduced.

The time expenditure and the costs for a successful ultrasound examination can be reduced.

In other respects, the one-off costs can be invested more effectively by using particularly high-quality equipment.

The subclaims show preferred further developments of the invention.

The determining of the information which represents the location and/or position of the transducer can take place for example by means of an optical camera.

This does not preclude the use of a stereo optical camera. This does preclude the use of stereoscopic sensors, mounted on the transducer. In this way the information representing the location/position of the transducer, is for example an image, a photograph, in particular a colour photograph, more preferably a moving image and most preferably a stereoscopy.

As such information is particularly easy for a person to interpret, an incorrect interpretation of the sonograms (ultrasound images) can be easily avoided in accordance with the invention.

The information, representing the position and/or location of the transducer, can be received for example, by means of a sensor which is movable, in particular automatically positionable, and/or stationary.

With a stationary arrangement, an average suitability of the perspective can be ensured in the best possible way, and an inadvertent maladjustment can be prevented.

By means of a movable/manually positionable arrangement of the sensor, a best possible perspective of the sensor on the body region of the patient examined by ultrasound, or respectively the transducer, can be ensured.

Automatic, in particular regulated positioning, can prevent circumstances occurring during the examination from impairing or spoiling the usability of a best possible documentation of the location/position of the transducer.

In order to further improve or respectively ensure the usability of the examination and documentation results, it can be automatically established in a further step that a determination of a location and/or position of the transducer is not possible at a second point in time.

For example, an automatic evaluation of the information representing the location and/or position of the transducer can be performed while the patient is still being examined by means of the transducer.

For example, it can be automatically determined that the transducer is not contained within the recorded data.

Alternatively, it can be determined that the perspective on the transducer and/or the examined body part of the patient is to be optimized.

To enable a remedial action to be taken, a signal is automatically output in response thereto.

The signal can be directed to a user (the doctor respectively the examining personnel).

The signal can be used, for example, to send the information (e.g. in text form and/or acoustically and/or optically) to the doctor/user that the perspective on the transducer is to be optimized or the transducer is to be brought into the detection range of the sensor used for documentation.

Alternatively or additionally, the signal may represent an instruction to adapt the distance between the sensor and the body part respectively between the sensor and the transducer or to optimize the recorded spatial angle.

Moreover, the signal can also represent information that the focus of the sensor used for documentation is to be adjusted.

The signal can also be used to automatically adapt the camera position, if the camera can be positioned by motor (automatically).

For this purpose, the camera can be automatically pivoted and/or can be repositioned in a translatory manner.

In particular, the camera position can be automatically varied within predefined limits to adjust best possible results and to maintain a corresponding position in the future.

The signal can, for example, refer to data from a second sensor for determining the location and/or position of the transducer.

In other words, the documentation material determined by the first sensor may be classified as less suitable than the documentation material determined by the second sensor.

In particular for a moving image sequence recorded over time by means of the transducer, the respectively best suited material can be automatically allocated for the documentation of the position/location of the transducer.

In other words, from the information recorded by means of the first sensor, data is only allocated to the ultrasound image material until the information determined by means of the second sensor is better suited for documentation and from then on the information received by means of the second sensor is allocated to the ultrasound image material, e.g. until, in the opposite direction, the suitability of the material which is received by means of the first sensor is better again.

If no documentation by means of a second sensor has been made, the use of the further and/or alternative (second) sensor can be initiated in response to the above-mentioned signal to determine the location and/or position of the transducer.

In other words, e.g. a further camera can be switched on and/or brought into position. The same applies for other sensor principles.

If applicable, the first sensor can be brought into a state of lower energy consumption (e.g. into a standby mode or switched off), since the information that can be determined by means of this first sensor in any case less suitable than that obtained by means of the further/alternative sensor.

Preferably, the data of an unsuccessful determination attempt can be automatically discarded.

In other words, data that document or attempt to document the location/position of the transducer in an unsuitable manner can be discarded, in particular automatically deleted, in order to release memory capacity. In particular, this can only be performed in response to a verification of the suitability of such data material that would be received by means of the second/further/alternative sensor.

This ensures that the most suitable data material for documenting the location/position of the transducer is always stored and allocated to the ultrasound image material.

Preferably, image material recorded for documenting the location and/or position of the transducer can be analysed using data technology, whereby in particular a comparison with a data technology reference can be carried out. For example, a computer-assisted (automatic) image analysis can be carried out, in which the image of the transducer and/or of the patient's body part are compared with corresponding references.

The reference may include corresponding characteristics of the transducer and/or of the patient's body part and a naming/other classification. Subsequently, the information material recorded for documenting the position/location of the transducer is classified automatically (e.g. “thigh”, “lower leg”, “knee”, or similar), for example the naming/other classification is associated with the data.

In this way, even if the specialist is not able to interpret the documentation on his own, a further assistance can be given, which enables a successful interpretation of the ultrasound images.

The above-mentioned classification moreover entails the advantage that particular terms (e.g. “thigh”, “lower leg”, “knee”, or similar) can be searched for in the examination material/within the documentation before the entire material is inspected. This also serves to automatically categorize all documented examinations, which can be helpful for retrospective evaluation of data in the field of medical research, for example.

Moreover, it can be automatically ensured that an examination has been carried out completely according to the order if the body part categories to be photographed are specified in an examination order and are documented as “completed” or respectively “open” in the course of the examination according to the invention.

The user of the device according to the invention can be informed of any missing categories if he wishes to terminate the examination despite the absence of a corresponding category of data material in the previous documentation.

Preferably, the position and/or the location of the transducer can be determined relative to a test piece and/or to a patient's body part.

In particular, standardized sensor positions/camera perspectives (e.g. distance of the sensor from the transducer and/or spatial angle of the main axis of the sensor, in particular with respect to a main axis of the transducer) can be agreed for the determination of the information representing the position of the transducer, the respective camera perspective can be determined automatically (sensor-based) and included in the documentation result. A possible camera perspective may, for example, be a camera mounted on the head of the user of the device according to the invention.

A further standardized camera perspective may be a top view (perpendicularly downwards). A still further camera perspective can be located substantially on the axis of the propagation direction of the ultrasound waves.

The above-mentioned camera positions can be used uniformly by a user group respectively across departments and therefore be interpreted particularly intuitively by medical specialists.

In particular, the data material (ultrasound images and information concerning location and/or position of the transducer) allocated to each other can be transmitted to a remote location by means of an intranet and/or the internet.

For example, the data can be stored in a data cloud.

Alternatively or additionally, an internet-based transmission to a monitor or other display device in another clinic respectively in another hospital or other location may be used.

The transmission can, for example, take place via the TCP-IP protocol and/or use a VPN connection. In particular, the data can be transmitted from the site of the examination directly into another city, country and/or continent, in order to combine worldwide expert knowledge for the interpretation/diagnosis and for teaching purposes in the best possible manner.

Especially in the case that an optical sensor system is used to document the ultrasound examination according to the invention, an allocation of the ultrasound images to the documentation data can be understood as “optical synchronization”.

A corresponding system synchronizes both the moving image data of the optical cameras and the ultrasound images of any type of ultrasound examination device.

The two data streams are merged and can subsequently be presented and/or saved simultaneously.

In particular, a split screen can be presented and/or a corresponding file can be saved.

In other words, a file is created for the divided optical display of the image material. Within the scope of the present invention, both data streams determined by means of conventional ultrasound apparatus and data streams recorded by means of conventional camera technology can be synchronized with each another, and independent apparatus including a camera input and a connection for an transducer can be used to realize the present invention.

The use of several camera sensors (stereo camera or similar) makes photographing of a three-dimensional representation of the area under examination possible.

The patient's body or body part(s) can be extracted from such a representation, whereby, for example, the background can be reduced respectively deleted.

The remaining image material (representing the body or body parts) can, for example, be allocated to a standard 3D body model or a rendered 3D representation of the body or body part actually examined. Those 3D models can be displayed as an anatomical view, in particular a skeletal-view, connective tissue-view, muscular system-view, arterial system-view, venous system-view, lymphatic system-view, nervous system-view, respiratory system-view, digestive system-view, endocrine system-view and/or urogenital system-view.

The position of the transducer can be determined by data technology (automatically) using the standard 3D body model or a rendered 3D representation of the body or body part actually examined by means of a comparison, and in particular markers on the transducer can be analysed to automatically determine the location/position of the transducer.

It is possible to zoom in and out of the created 3D models representing the body or body part of the examined patient to give a clearer picture of the area under investigation.

The 3D model can be viewed in all spatial directions including a cross section view.

The 3D body can reflect the movement of the recorded person during the examination.

The above-mentioned features of the present invention do not preclude that examination modalities of the ultrasound examination, in particular the location of a focus during the creation of the ultrasound images, are stored together with the data material determined according to the invention (for the location (deep) coding of the examined “lesion” in the ultrasound image, e.g. the tumour is at 2 cm depth to the skin)).

According to a second aspect of the present invention, a device for supporting an evaluation of an ultrasound-based examination is proposed. The device comprises a data input which, for example, comprises an HDMI- and/or DVI- and/or USB-port.

Video data streams can be received from the ultrasound apparatus via the HDMI or DVI port or any ports provided by the manufacturers. Optical video cameras can be connected via the USB port or the USB ports or any ports provided by the manufacturers.

The device furthermore comprises, an evaluation unit which may comprise a programmable processor, a microcontroller, an electronic control device or similar, for example. Finally, a data output is also provided, which can be configured as a card reader, for example. Especially SD or MMC cards can be written via the card reader.

The data output may alternatively or additionally include a USB port for connecting a storage medium and/or an HDMI or DVI port or any ports provided by the manufacturers for connecting an additional monitor and alternatively or additionally an ethernet port (RJ-45) for sending the data material in an intranet- and/or internet-mediated manner.

Optionally an operating element configured in hardware (e.g. a button) and/or software, by means of which the recording of ultrasound image material can be started optionally simultaneously with the documentation according to the invention of the location/position of the transducer, can be provided. In this way it can be prevented that inadvertently one forgets to launch the documentation in the manner according to the invention. The evaluation unit is arranged, by means of the data input in the execution of the ultrasound-based examination by means of an transducer at a point in time, to determine information representing a location and/or position of the transducer at the point in time. For this purpose, the data input receives the signals of the transducer and an additional sensor (e.g. for example an optical camera). By means of the data output, the evaluation unit can automatically allocate an image received at the point in time by means of the transducer and information representing the location and/or position of the transducer to one another. In this way, the device according to the invention is arranged to realize the features, feature combinations and advantages of the method according to the invention evidently in a corresponding manner in such a way so that to avoid repetitions reference is to be made to the above comments.

### BRIEF DESCRIPTION OF THE FIGURES

Further details, advantages and features of the present invention will emerge from the following description of example embodiments with the aid of the drawings. There are shown:

FIG. 1 a schematic illustration of the components of a first example embodiment of a device according to the present invention;

FIG. 2 a schematic illustration of the components of a second example embodiment of a device according to the present invention;

FIG. 3 a schematic illustration of the components of a third example embodiment of a device according to the present invention;

FIG. 4 a schematic illustration of the components of a fourth example embodiment of a device according to the present invention; and

FIG. 5 a flow diagram illustrating steps of an example embodiment of a method according to the invention for supporting an evaluation of an ultrasound-based examination.

### DETAILED DESCRIPTION

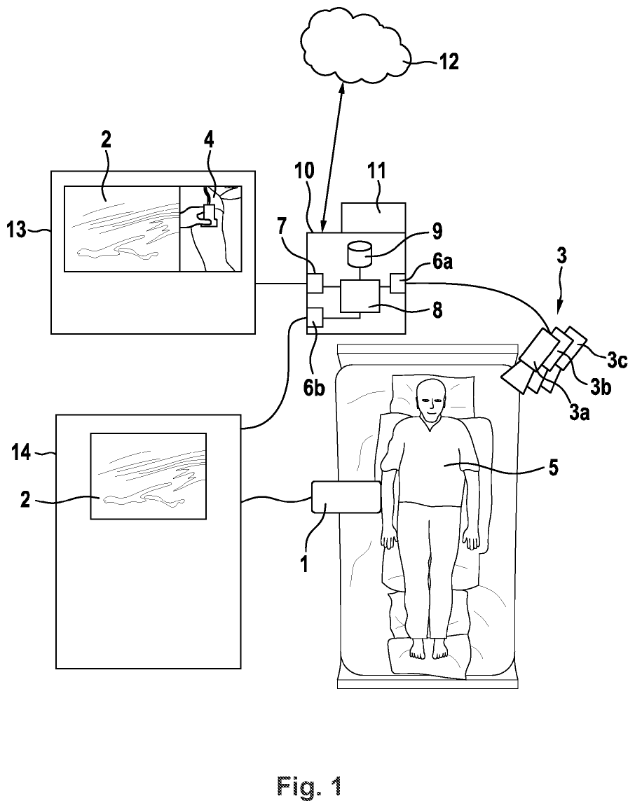

FIG. 1 shows a first example embodiment of a device according to the invention in the embodiment of an example embodiment of a method according to the invention. A patient 5 is to be examined by means of an transducer 1, which is connected in terms of information technology to an ultrasound apparatus 14. Ultrasound images 2 can be presented on the ultrasound apparatus 14. Such a device is prior art. In accordance with the invention, the image material 2 is forwarded to a device 10 according to the present invention or respectively into its data input 6b. From the data input 6b the image material 2 arrives at a programmable processor 8 as an evaluation unit, which is also connected in terms of information technology to a data memory 9. In the data memory 9 instructions can be stored, which by carrying out on a programmable processor 8 initiate the steps of an example embodiment of a method according to the invention. The patient 5 is also situated in in the detection range of a sensor arrangement 3, which has three optical camera 3a, 3b, 3c and is connected in terms of information technology to a data input 6a of the device 10. The cameras 3a, 3b, 3c record the patient from different perspectives, whereby on the one hand a permanent detection of the transducer 1 as well as the respective body part of the patient 5 can be ensured and, on the other hand, a stereoscopic (depth image/location coding) detection of the situation is possible. The data determined using the data input 6a and data input 6b can be stored in the data memory 9, extracted via a USB port 11 and/or stored in a data cloud 12. Via a data output 7, which can be configured for example as an HDMI- or DVI-port, the ultrasound image 2 can be output in a split screen presentation with the optical images 4. This concerns a respective video image (moving image) presentation. The ultrasound images 2 and the optical images 4 are synchronized with one another in such a way that the presentation always indicates the data which are recorded at an identical point in time. In this way, the presentation of the optical images 4 supports the user in the interpretation of the sonogram. In the illustrated example embodiment, the display device 13 is embodied as an external monitor which is not necessarily to be understood as a component of the device 10 according to the invention.

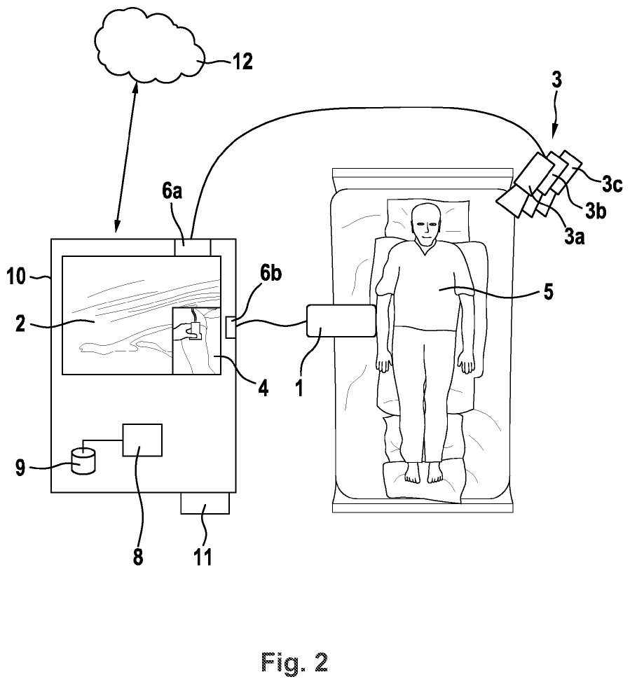

FIG. 2 shows a second example embodiment of a device 10 according to the invention, in the embodiment of an example embodiment of a method according to the invention. To a large extent, the components correspond to those of the first embodiment (FIG. 1). However, the second example embodiment combines the display device 13, the ultrasound apparatus 14 and the device 10 according to the invention in a shared housing. Accordingly, the device 10 according to the invention now has, in addition to the data inputs 6a and 6b the means for displaying the ultrasound images 2 and the optical images 4. An internal data memory 9 and a USB port 11 are also provided in the housing of the device 10, as is a data interface for the data exchange with the data cloud 12. The remaining components correspond to the embodiment illustrated in FIG. 1.

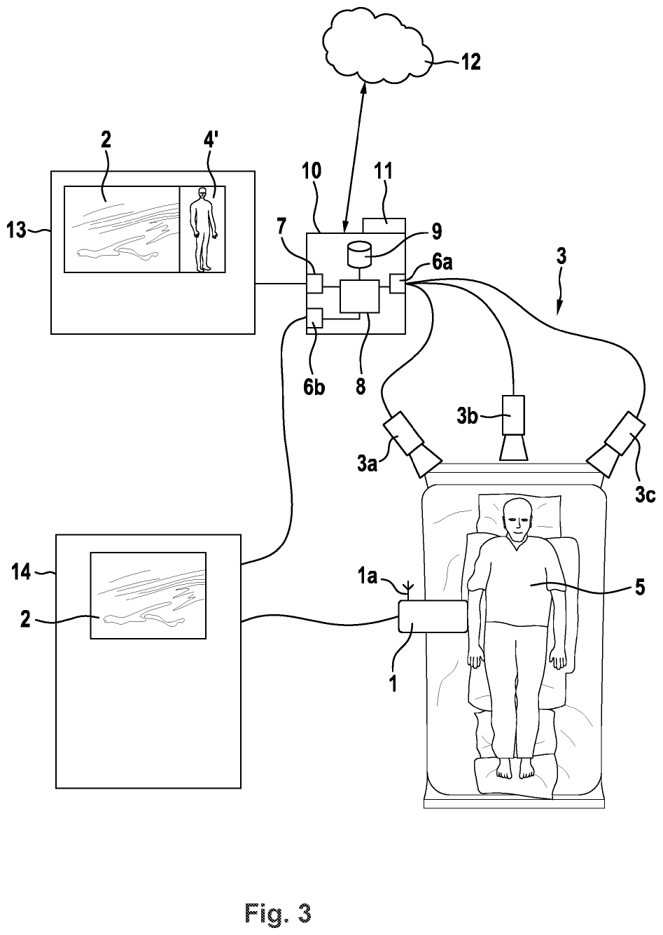

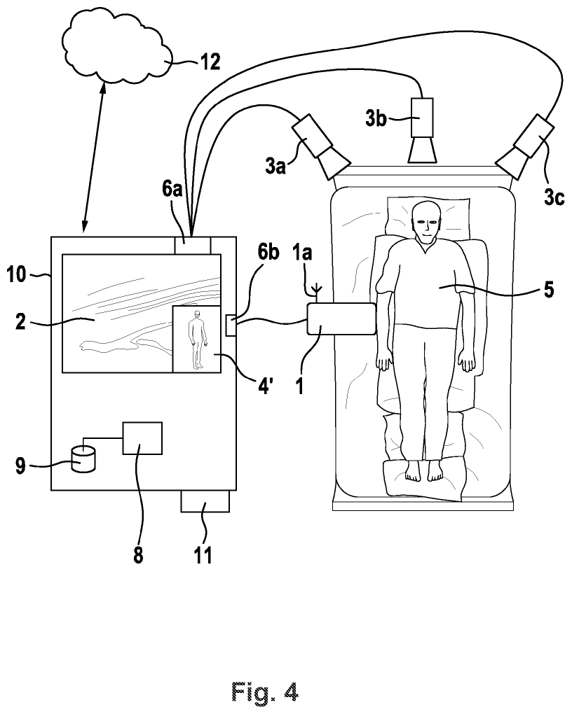

FIG. 3 shows a third example embodiment of a device according to the invention, in the embodiment of an example embodiment of a method according to the invention for the creation of a three-dimensional view, which is determined in a sensor- and camera-based manner. The components broadly correspond to those of the first embodiment (FIG. 1). However, instead of displaying camera images on the external display device 13, a three-dimensional model 4′ is presented alongside the ultrasound images 2. FIG. 3 and FIG. 4 include the recording of the patient and technical conversion into a 3D model exactly reproducing the patient or the part of his body. Hereby, the 3D model can be reproduced from any desired perspective which does not have to correspond to the actual perspective of the examiner, but reproduces the actual location/time coding. Hereby, e.g. an image collision (blind angle) which could occur through the pure camera version, can be prevented in the reproduction of the images presented by a device according to the invention. Furthermore, the ultrasound image can be placed virtually into the 3D model, therefore superimposed, in order to allow an absolute location coding. In order to enable a most exact positioning of the transducer 1 as possible, it has an electromagnetic transmitter (motion capture marker and inertial measurement unit (IMU)) 1a or a marker, which can be optically detected by means of the cameras 3a, 3b, 3c and can be used to determine information representing the position/location of the transducer 1. The transmitter 1a can also be used to determine the location of the transducer 1 by means of the device 10 according to the invention.

FIG. 4 shows a fourth example embodiment of a device according to the invention in the embodiment of an example embodiment of a method according to the invention. The components broadly correspond to those of the third embodiment (FIG. 3), wherein, however, the components shown in FIG. 3, ultrasound apparatus 14, display unit 13 and device 10 are arranged in a shared housing, so that the device 10 according to the invention comprises means for the locating of the transducer 1 as well as means for displaying the ultrasound images 2 and the three-dimensional model 4′ and the data interface to a cloud 12.

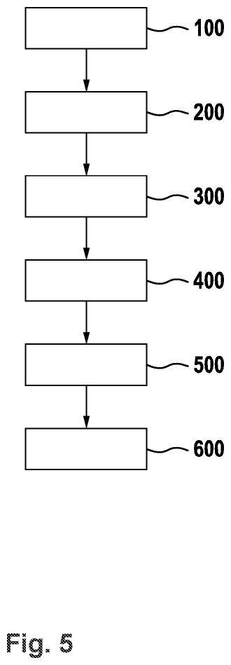

FIG. 5 shows steps of an example embodiment of a method according to the invention for supporting an evaluation of an ultrasound-based examination. In step 100, the ultrasound-based examination is carried out by means of an transducer at a certain point in time. In other words, an transducer is used, in order to create a sonogram of a patient's body part. In step 200, information representing a location and a position of the transducer at the same time is determined. In order to give the specialist a best possible support in the interpretation of the sonogram, the sonogram is automatically allocated in step 300 to the information which represents the position and location of the transducer. This step could also be designated as synchronization. In step 400 the image recorded at the point in time by means of the transducer and the location and position of the transducer are transmitted to a data cloud by means of the intranet and of the internet, from which data cloud the received data can be interpreted by a remotely situated specialist on a remotely positioned display device, or stored for a later analysis. In step 500 it is established that a determining of a location and position of the transducer at a second point in time is not possible. This may be due to the fact that the examiner shadows the transducer with his body with respect to a location/position in a representing manner for the receiving of the information. Therefore, in step 600, a signal is output to the user that the current perspective of the image sensor which in use is not suitable to detect the transducer and its location/position, and the user should either choose a different perspective for the sensor or should himself move to a different position, so as not to spoil the recording of the location/position data of the transducer. The signal can also be used in order to automatically bring a camera, which is movable by a motor, to a better suited position and therefore to automatically optimize its perspective onto the transducer.

### LIST OF REFERENCE NUMBERS

1 transducer
1a transmitter
2 ultrasound image
3 sensor arrangement
3a, 3b, 3c cameras
4 camera image
4′ 3D model
5 patient
6a, 6b data input
7 data output
8 programmable processor
9 data memory
10 device
11 USB port
12 data cloud
13 display unit
14 ultrasound apparatus
100, 200, 300, 400, 500, 600 method steps

1. A method for supporting an evaluation of an ultrasound-based examination comprising:
carrying out the ultrasound-based examination by means of a transducer at a point in time,
determining information representing a location and/or position of the transducer at the point in time and
automatic allocating
of an image, recorded at the point in time by means of the transducer to
the information representing the location and/or position of the transducer at the point in time.

2. The method according to claim 1, wherein the determining of the information representing the location and/or position of the transducer takes place by means of an optical camera, in particular as an image, preferably as a moving image.

3. The method according to claim 2, wherein the camera is arranged so as to be fastened displaceably, in particular so as to be automatically positionable, or stationary.

4. The method according to claim 1, further comprising
establishing that the determining of a location and/or position of the transducer is not possible at a second point in time and in responding thereto
emitting of a signal.

5. The method according to claim 4, wherein the signal
refers to data of a second sensor for determining the location and/or position of the transducer, and/or
automatically initiates a use of a further and/or alternative sensor for determining the location and/or position of the transducer and/or
automatically initiates a position/alignment change of the second sensor for determining the location and/or position of the transducer and/or
automatically discards data of an unsuccessful attempt at determination.

6. The method according to claim 1, wherein for determining the information representing the location and/or position of a transducer image material is analysed and is automatically compared with a data technology reference, in particular comprising an image data bank, in order to draw a conclusion regarding the location and/or position of the transducer and/or of a patient.

7. The method according to claim 1, wherein the image recorded by means of the transducer is a component part of a moving image sequence.

8. The method according to claim 1, wherein the location and/or position of the transducer is determined relative to a test piece and/or to a part of a body of a patient.

9. The method according to claim 1, wherein the allocating comprises a synchronisation of respective data of a recorded image and of the information representing the location and/or position of the transducer.

10. The method according to claim 1, further comprising
transferring of the image recorded at the point in time by means of the transducer, and of the location and/or position of the transducer by means of an internet and/or by means of an intranet.

11. The method according to claim 1, further comprising
detection by sensor of a marker on the transducer and
presentation of a display representing the location of the transducer by means of a detected marker.

12. A method for supporting an evaluation of an ultrasound-based examination of a patient comprising the steps:
carrying out the ultrasound-based examination by means of a transducer at a point in time,
determining information representing a location and/or position of a transducer at the point in time, wherein for determining the information representing the location and/or position of the transducer image material is analysed and is automatically compared with a data reference in order to identify the location and/or position of the patient,
and
automatic allocating
of an image, recorded at the point in time by means of the transducer to
the information representing the location and/or position of the transducer at the point in time.

13. A device for supporting an evaluation of an ultrasound-based examination comprising:
a data input,
an evaluation unit and
a data output, wherein
the evaluation unit
is arranged by means of the data input, in carrying out of the ultrasound-based examination by means of a transducer at a point in time to determine information representing a location and/or position of the transducer at the point in time and
is arranged by means of the data output
to automatically allocate with respect to one another an image recorded at the point in time by means of the transducer and
the information representing the location and/or position of the transducer.
